(19) United States
(12) Patent Application Publication
(10) Pub. No.: US 2020/0075174 A1
(43) Pub. Date: Mar. 5, 2020

(54) METHOD AND SYSTEM FOR COLLECTING INFORMED CONSENT OF A PATIENT

(71) Applicant: Pascal GLEYZE, Andolsheim (FR)

(72) Inventor: Pascal GLEYZE, Andolsheim (FR)

(21) Appl. No.: 16/462,705

(22) PCT Filed: Nov. 13, 2017
(86) PCT No.: PCT/FR2017/053081

§ 371 Date: May 21, 2019

(30) Foreign Application Priority Data
Nov. 21, 2016 (FR) FR 16 61295

(51) Int. Cl. G16H 70/00; G16H 40/20; G16H 80/00; G06Q 10/10

(52) U.S. Cl. CPC G16H 70/00; G06Q 10/10; G16H 80/00; G16H 40/20

(57) ABSTRACT

A system for obtaining a patient's informed consent to establish a computer session between a patient's computer equipment, command the transmission of the digital multimedia files to the patient's computer equipment, record at least one piece of information concerning opening a session between the patient's computer equipment and the server, as well as information transmitted by the patient's computer equipment, said system further comprising a plurality of material supports each having an identical information area, a patient/practitioner signature area, and a couple formed by a unique identifier and a password, for activating the step of opening a secure session between the server and computer wherein said program means for controlling the operation of the system include recording means in a memory space associated with the unique identifier used for opening the secure session time-stamped information relating to processing digital multimedia files on the computer corresponding to said secure session.

### BACKGROUND OF THE INVENTION

This invention concerns the field of methods and information systems to automate the organization of the procedure for informing a patient prior to the initiation of a medical procedure, and the management of the establishment of proof of the patient's informed consent.

It concerns in particular the inception on paper or electronic media of a specific therapeutic action, such as the patient's ideal theoretical informative consultation during a care procedure. The implementation of this therapy is considered as an element of care.

In order to ensure the patient's conscious and well-understood involvement prior to the administration of a medical or surgical procedure, the practitioner responsible for the management is required to ensure:
in a first step, the provision of fair, clear information adapted to the patient's level of understanding and whose content, formatting and administration must be compatible with the expected care principle.
in a second step, the collection of the patient's consent, which must be not only informed by the information previously provided, but also free of any pressure or constraint.

The free and informed nature of the consent implies that a reflection period must be provided between the time of information and the time of consent.

The content of the prior information that the practitioner provides to the patient is almost exhaustive and personalized as for the risks inherent to the recommended intervention.

To the extent that the information must necessarily relate to “normally foreseeable” risks, i.e. risks known in the light of the state of science, either because they are “serious” or because they are “frequent”, only minor exceptional risks are ultimately exempt from the obligation.

The information provided to the patient is not limited to standard information on the risks inherent to the procedure. The practitioner must carry out a personalised assessment of the benefits and risks of the proposed procedure, taking into account the specific characteristics of the patient (state of health, age, profession, life style, family setting, etc.).

The practitioner must also inform the patient of the different therapeutic alternatives likely to remedy his pathology, leaving the latter free to choose in light of the benefit/risk assessment.

The information owed to the patient must be provided orally during a consultation scheduled for this purpose, including a personal interview, which must take place in complete confidentiality in the doctor's office and under no circumstances in a corridor or waiting room.

In the event of a complaint from the patient, it is the responsibility of the health professional or health care institution responsible for the care to provide proof that the information has been properly provided. It is therefore essential, from a forensic point of view, to pre-constitute evidence in this area.

Indeed, when faced with a patient claiming not to have received fair, clear and appropriate information, it can be complicated to demonstrate not only the reality of the information, but also its quality.

In addition to the forensic implications, the patient information process improves the psychological consideration of the patient, which is an integral part of the quality of care.

The state-of-the-art is known from US2012310670 patent application describing an automated process for verifying a patient's informed consent. It includes multimedia resources and interactive presentation means for providing standardized information to a patient about a specific medical procedure in medical procedures or clinical trials.

The presentation is patient-specific, using the patient's medical history to personalize the information to be provided regarding the risks and expected outcomes of the proposed medical procedure.

The presentation is provided on a computer to a patient who interacts with the informed consent presentation to ask and answer questions, indicating their ability to make a decision and to verify understanding of the content of the presentation.

The patient's consent may be recorded in an audio or video recording to more accurately capture the patient's consent and verify that the patient has consented to the proposed medical procedure.

Patent application US2013073310 describes an interface for the collaboration between patients, health professionals and third parties. Patients, healthcare professionals and third parties can share information about medical procedures, products and services using computer-based and web-based human-machine interfaces that implement a highly interactive and personalized mobile healthcare interface. This prior art document describes a solution that allows health professionals to obtain informed consent from patients and to improve patient compliance with medical instructions and allow health professionals to monitor overall compliance.

Patent U.S. Pat. No. 5,799,282 describes a method for the certified establishment of informed consent for a medical procedure. The patient interacts with a video training system until all required information is successfully mastered.

Training techniques that trigger measurable patient behaviours such as a guide to discern the patient's level of knowledge are used.

Certification is granted only when the measurable behaviour approximately coincides with legal and medical standards to establish informed consent. The system is able to adapt to different medical procedures, patient attitudes and knowledge bases, while maintaining a level of overall consistency. In one embodiment, a minimum probability of comprehension can be pre-set, and the training procedure will continue until the actual statistical probability of understanding is at least equal to the predefined minimum.

The international patent application WO2006047832 describing the computerized process for obtaining a patient's informed consent, in the occurrence of an event having a plurality of aspects of the event, is also known. The method involves building a database containing event information related to each aspect of the event, and presenting some of this information to a patient to verify the patient's understanding of the event. The method includes an inquiry step to verify whether the person has understood an aspect of the event or requires additional information. If the person indicates that the event information has been heard, the process commands the display of at least one question to test the reality of understanding.

The solutions proposed in the prior art have several drawbacks.

First, they do not automate the proof of compliance of the entire information process, involving an oral discussion between the practitioner and the patient.

Second, prior art solutions do not provide relevant digital evidence of the nature of the information provided to the patient, which has resulted in the registration of evidence of informed consent.

Moreover, they do not provide complete, updated and validated information that meets the criteria and procedures for administering this information. As such, they are expert control systems but no content corresponding to the implementation of care is specified or proposed. There is no provision of content or commitment to make available an active therapeutic ingredient corresponding to current regulations.

### SUMMARY OF THE INVENTION

A system for obtaining informed consent from a patient having a server including a memory for recording a plurality of digital multimedia files and program means for controlling the operation of said system, so as to:
establish a computer session between a patient's computer equipment equipped with a browser and said server,
command the transmission of the digital multimedia files to the patient's computer equipment,
record at least one piece of information concerning the opening of a session between the patient's computer equipment and the server, as well as information transmitted by said patient's computer equipment via a digital form, characterized in that it also includes a plurality of material supports, such as a paper or computer support (for example a digital page displayed on a tablet or a computer) each having an identical information area, a patient signature area, a signature area for the practitioner and a couple formed by a unique identifier and a password corresponding to the digital information previously stored in a memory of said server, for activating the step of opening a secured session between the server and a computer equipment, and in that said program means for controlling the operation of the system include means for recording in a memory space associated with the unique identifier used for opening the secured session time stamped information relating to the processing of digital multimedia files on the computer equipment corresponding to said secured session.

Preferably, the material supports consist in a prescription book containing a plurality of detachable sheets.

Advantageously, these detachable sheets form pairs of sheets with the same unique identifier.

According to a particular embodiment, said program means for controlling the operation of the system include means for encrypting information associated with an identifier and storing in a memory, at the end of a session, time-stamped information in an encrypted form only.

The invention also concerns a method for obtaining a patient's informed consent, comprising the steps of establishing a computer session between a patient's computer equipment equipped with a browser software and a server, and the steps of transmitting to said patient's computer equipment digital multimedia files and recording at least one piece of information concerning the opening of a session between the patient's computer equipment and the server, as well as information transmitted by said patient's computer equipment via a digital form, characterized in that it includes an initial step of communication by the practitioner and the patient of a material support comprising a unique identifier, corresponding to an identifier previously recorded in the server's memory, the practitioner and the patient each keeping a copy of said material support signed by each of the parties and in that said program means for controlling the operation of the system include means for recording in a memory space associated with the unique identifier used to open the secure session time-stamped information relating to the processing of digital multimedia files on the computer equipment corresponding to said secure session.

Advantageously, said program means for controlling the operation of the system include means for encrypting information associated with an identifier and storing in the memory, at the end of a session, time-stamped information in an encrypted form only.

According to a preferred embodiment, the method includes steps of transmitting an activated digital form after the transmission of the multimedia digital files corresponding to the act requiring informed consent, said digital form including means for entering information provided by the patient and recorded in the memory in relation to said unique patient identifier.

### BRIEF DESCRIPTION OF THE DRAWINGS

The present invention will be best understood upon reading the following detailed description of a non-limiting exemplary embodiment, while referring to the appended drawings, wherein:

FIG. 1 is a schematic view of a functional architecture according to the invention.

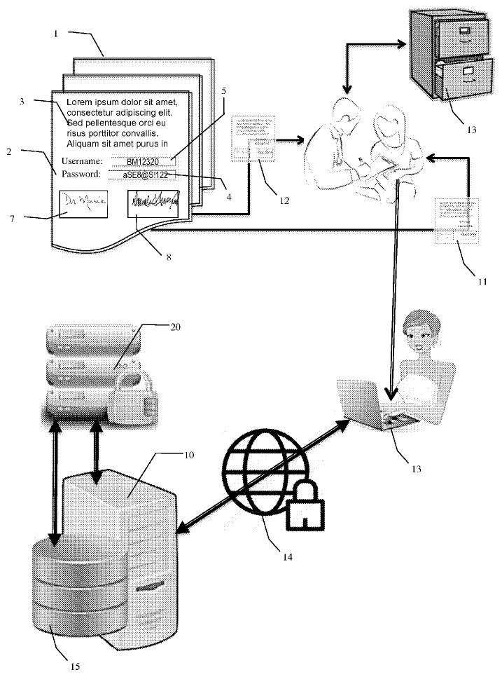

### DETAILED DESCRIPTION

The practitioner has a prescription book (1) for the prescription of information relating to a healthcare procedure. This prescription book is an innovation in that it makes it possible to prescribe a treatment whose active principle is based on an information content and not on a molecule or a physical therapeutic action. This prescription book (1) consists in the example described above of a set of carbonless sheets (2) with text areas (3) for information on the context of the patient information process and the organization of the collection of informed consent.

The two paired sheets (2) each have a unique identifier (4) and a password (5).

When printing a sheet, the manufacturing process of the prescription book (1) includes a step of importing a series of unique identifiers calculated by a server (10). These unique identifiers are also stored, in the example described, in a secure memory (20). The information constituted by the series of unique identifiers can be associated with information uniquely identifying the prescription book, with a step of entering a prescription book identifier on the prescription book.

The sheets (2) also present two areas (7, 8) to receive the signatures of the practitioner on the one hand, and of the patient on the other hand, after the completion of a consultation during which the practitioner orally exposes the context of the proposed medical procedure, questions the patient about its history, his enquiries and more generally about all the questions the patient has.

At the end of this interactive phase carried out face-to-face between the practitioner and the patient, both sign a pair of sheets, one of the signed sheets (11) being kept by the patient, the other signed sheet (12) being kept by the practitioner, for example in an archive cabinet (13).

The patient can then, according to his availability and at his own pace, connect to the server (10) using any computer equipment (13) via a browser software that opens a secure https session. Access to the server (10) is carried out in the example described anonymously, using the unique identifier and password on the sheet (11) given to the patient. If necessary, the system can implement an IP address anonymization server (14) to avoid the recording of any personal data.

The anonymization server (14) can be an entity that provides network access to the user and assigns him an identifier to communicate on the network. The anonymization server (14) is, for example, a mobile network operator, a mobile virtual network operator or an Internet service provider (ISP) to which the user has a subscription. The anonymization server (14) can also be a server of a specialized and recognized private organization, the anonymization server (14) using a deterministic encryption algorithm to encrypt the user's identification data with the AK anonymization key. This deterministic encryption algorithm is a cryptosystem that always produces the same encrypted text for the same data. The collection server (10) can therefore observe the behaviour of this encrypted identifier received from the anonymization server (14) over time. Through the profile data received for this encrypted identifier, the collection server (10) can statistically analyze the received encrypted identifiers to refine the user profile, without knowing his identity.

When collecting information from the patient's computer equipment (13) by the server (10), said anonymization server (14) performs the following steps:
encryption of the data to be collected with a confidentiality key shared between said terminal (13) and the server (10),
transmission of the data to be collected in encrypted form and data likely to contribute to the identification of the patient to an anonymization server (14) interposed between the terminal (13) and the server (10),
encryption of data capable of contributing to the identification of the patient with an anonymization key of said anonymization server (14) before relaying the collected data and encrypted identification to said server (10).

Once the patient has logged in securely, he or she accesses a set of digital multimedia files stored in a multimedia memory (15).

This includes, for example, graphic or video presentations concerning the anatomical context, the didactic presentation of a surgical procedure, post-operative risks, etc.

When an authorized patient connects to the server (10) through a log-in procedure, the system recognizes the patient's unique identifier and records in a log stored in the encrypted memory (20) the time stamped details of the information accessed.

The multimedia files recorded in the database (15) are for example in XML format, incorporating a script (or program) that runs each time a web page, containing this script, is displayed on the patient's equipment (13), i.e. is downloaded.

A copy of each of the files is stored in the memory (20), in an unalterable form, in connection with an electronic signature.

In this case, the scripts generated by the server (10) and inserted in the XML document allow to count the number of accesses to the site via the search tool in which it is referenced. Indeed, when a patient clicks on a link to the client site in the list of answers proposed by the search tool, this tool records data relating to the patient and his consultation.

The information recorded in relation to the patient's unique identifier in the encrypted memory (20) includes all or part of:
the electronic signature of the downloaded multimedia file,
the time and date of the opening of each visit session and the possible downloading of the downloadable documents presented in the space allocated to the patient code,
the time and date of activation of a link in the downloaded file, as well as the identifier of that link,
the answers entered by the patient in a file containing a “QUIZ” form,
the messages sent allowing the supervision of the actions carried out by the patient on each page, information and media included in the space (message of reading reminder, alerts on actions to be carried out in connection with the communicated information, etc.)

The server conditionally offers an acknowledgement form allowing the patient to confirm his/her informed consent.

This form is only sent if the patient's path complies with a minimum path, necessary for full patient information. This path is calculated according to the information stored in the memory (20) in relation to the patient's unique identifier.

1. A system for obtaining a patient's informed consent including a server including a memory for recording a plurality of digital multimedia files and program means for controlling the operation of said system, so as to:
establish a computer session between a patient's computer equipment equipped with a browser and said server,
command the transmission of the digital multimedia files to the patient's computer equipment,
record at least one piece of information concerning the opening of a session between the patient's computer equipment and the server, as well as information transmitted by the patient's computer equipment via a digital form,
said system further comprising a plurality of material supports each having an identical information area, a patient signature area, a signature area for the practitioner and a couple formed by a unique identifier and a password corresponding to the digital information previously stored in a memory of said server, for activating the step of opening a secure session between the server and computer equipment and wherein said program means for controlling the operation of the system include means for recording in a memory space associated with the unique identifier used for opening the secure session time-stamped information relating to the processing of digital multimedia files on the computer equipment corresponding to said secure session.

2. The system for collecting a patient's informed consent according to claim 1, wherein said material supports consist in a prescription book containing a plurality of detachable sheets.

3. The system for collecting a patient's informed consent according to claim 2 wherein said detachable sheets form pairs of sheets bearing the same unique identifier.

4. The system for collecting a patient's informed consent according to claim 1 wherein said program means for controlling the operation of the system include means for encrypting information associated with an identifier and for storing in a memory, at the end of a session, time-stamped information in an encrypted form only.

5. A method for obtaining a patient's informed consent comprising the steps of establishing a computer session between a patient's computer equipment equipped with browser software and a server, and the steps of transmitting to said patient's computer equipment digital multimedia files and recording at least one information concerning the opening of a session between the patient's computer equipment and the server, as well as information transmitted by said patient's computer equipment via a digital form, said method further comprising an initial step of communication between the practitioner and the patient of a material support comprising a unique identifier, corresponding to an identifier previously recorded in the server memory, the practitioner and the patient each keeping a copy of said material support signed by each of the parties, and in that said program means for controlling the operation of the system include means for recording in a memory space associated with the unique identifier used to open the secure session time-stamped information relating to the processing of multimedia digital files on the computer equipment corresponding to said secure session.

6. The method for collecting a patient's informed consent according to claim 5, wherein said program means for controlling the operation of the system include means for encrypting information associated with an identifier and for recording in a memory, at the end of a session, time-stamped information in an encrypted form only.

7. The method for collecting a patient's informed consent according to claim 5, further comprising steps of transmitting an activated digital form after the transmission of the multimedia digital files corresponding to the medical procedure requiring the informed consent, said digital form comprising means for entering information provided by the patient and recorded in the memory in relation to said unique identifier of the patient.
